(19) United States
(12) Patent Application Publication
(10) Pub. No.: US 2010/0168256 A1
(43) Pub. Date: Jul. 1, 2010

(54) USE OF PHOSPHORIC TRIAMIDES IN CLEANER AND HYGIENE APPLICATIONS

(75) Inventors: Frank Rittig, Mannheim (DE); Volker Braig, Weinheim-Luetzelsachsen (DE); Oliver Huttenloch, Ispringen (DE); Patrick Deck, Mannheim (DE); Johan Ebenhoech, Mannheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/438,005

(22) PCT Filed: Aug. 8, 2007
(86) PCT No.: PCT/EP2007/058240

§ 371 Date: Feb. 19, 2009

(30) Foreign Application Priority Data
Aug. 24, 2006 (EP) 06119500.4

(51) Int. Cl. A01N 25/00; C09B 67/42; C11D 3/36; A61K 8/42; A61Q 13/00

(52) U.S. Cl. 514/788; 8/584; 510/467; 510/278; 510/240; 510/238; 512/26

(57) ABSTRACT

The present invention describes the use of phosphoric triamides in cleaner and hygiene applications.

Phosphoric triamides are described in U.S. Pat. No. 4,530,714. The use of these compounds is likewise described therein—upon use in crop growth media, these contribute in the field to the nitrogen content in the soil being retained.

U.S. Pat. No. 5,770,771 discloses a multistage process for the preparation of N-hydrocarbyl-thiophosphoric triamides, e.g. of N-(n-butyl)thiophosphoric triamide.

WO 2006/010389 A1 deals in turn with the use of phosphoric triamides. Here, a number of uses is mentioned: the avoidance of nitrogen losses in the case of the use of nitrogen-based fertilizers, the avoidance of the ammonia nuisance in animal stalls, the use of urea as feed additive in animal nutrition, and also medical applications.
The avoidance of nitrogen losses in the case of the use of nitrogen-based fertilizers is accordingly achieved by the enzymatic urease-catalyzed urea hydrolysis being suppressed—if appropriate in combination with a parallel limitation of the nitrification. This is said to avoid high ammonia concentrations in the soil, which can adversely affect germination and the emergence of seedlings.
By inhibiting the enzymatic urease-catalyzed urea hydrolysis, the aim is likewise to avoid the sometimes considerable ammonia nuisance in stalls, which can adversely affect the development and the growth of the animals.
And also, according to WO 2006/010389 A1, in this way, the partial substitution of the high-value protein-rich plant nutrition of animals through so-called “non-protein nitrogen compounds” is possible. Here, urea can be used if it is possible to control the urease-catalyzed urea hydrolysis occurring in the rumen of the animals in such a way that the released ammonia rates can be processed immediately by microorganisms that are present to give microbial protein and thus can trigger no toxic reactions.
In the medical sector, the urease inhibitors are proposed for the prophylaxis or treatment of disorders or diseases which are directly or indirectly induced or encouraged by urease activity. Examples are catheter encrustations, ulcerous stomach and intestinal diseases, urolithiasis, pyelonephritis, nephrolithiasis, ammonia encephalopathy, hepatic encephalopathy, hepatic coma, urinary tract infections and gastrointestinal infections.

DE 102 52 382 A1 likewise deals with the avoidance of nitrogen losses in the case of the use of nitrogen-based fertilizers, the avoidance of the ammonia nuisance in animal stalls and the use of urea as feed additive in animal nutrition. In this connection, it describes phosphoric ester diamides as extremely effective urease inhibitors. It likewise describes N-(n-butyl)thiophosphoric triamide as a derivative of phosphoric triamide, although it is described as being relatively susceptible to hydrolysis. DE 102 52 382 A1 teaches the use of tetraaminophosphonium salts as particularly readily suitable compounds for the specified applications.

U.S. Pat. No. 6,869,923 describes a perfume composition which can be used in the sanitary sector in cleaners. However, this composition has the disadvantage that a user who only wishes to avoid the odor or urine during and after cleaning does not have this option, but can conceal the odor only by means of another, generally perceived as pleasant.

U.S. Pat. No. 6,376,457 also describes a perfume composition for use in cleaners in the sanitary sector. Here too, the user does not have the option of avoiding the unpleasant odor of urine—he can conceal it merely by means of a more intensive—again generally perceived as pleasant—odor.

U.S. Pat. No. 6,625,821, which deals essentially with a dispersion device for cleaners and fragrances in toilets, likewise only describes the use of fragrances for solving the problem of urine odor in the sanitary sector.

This gives rise to the object of avoiding the odor of urine during the cleaning of surfaces contaminated with urine, and to the object of preserving the cleaned surfaces such that, upon renewed exposure to urine, they only absorb and/or release the corresponding odor after a delay, and ideally not at all.

Surprisingly, these objects are achieved by the composition according to claims 1 to 14, the dosing devices according to claims 15 and 16, the kit of parts according to claim 17, the cleaning device according to claim 18 and the use of this according to claims 19 and 20.

A composition comprising at least one N-alkylthiophosphoric triamide and at least one surfactant selected from the group consisting of anionic surfactants, cationic surfactants and betaine surfactants achieves the set objects according to the invention.

In this connection, preference is given to a composition in which the (total) amount of N-alkylthiophosphoric triamide(s) is 0.01 to 75 mass %, particularly preferably 0.1 to 50 mass % and very particularly preferably 0.25 to 20 mass %. A composition as described in which the (total) amount of N-alkylthiophosphoric triamide(s) is 0.5 to 10 mass % is most preferred. The best cleaning and preserving effects are achieved when the composition comprises the one or more N-alkylthiophosphoric triamide(s) in amounts of, for example, 0.6, 0.8, 1, 2, 5 or 8 mass %.

A composition as described, in which the at least one surfactant is present in a (total) amount of from 0.01 to 99 mass %, is preferred, more preferred is a composition in which the at least one surfactant is present in a (total) amount of from 0.5 to 50 mass % and even more preferred from 1 to 25 mass %. A composition as described in which the at least one surfactant is present in a (total) amount of from 1 to 15 mass % is most preferred. Here, the best cleaning and preserving effect arises when the composition comprises one or more surfactants in a (total) amount of, for example, 2, 5, 8, 10 or 12 mass %.

Here, the statement “(total) amount” is to be understood as meaning that in cases where only one n-alkylthiophosphoric triamide or only one surfactant according to the invention is present in the composition, the amount of this substance is to be taken into consideration, whereas in cases where two or more n-alkylthiophosphoric triamides or two or more surfactants according to the invention are present, the sum of each of these is to be taken into consideration. Thus, if the compound comprises x mass % of anionic surfactant and y mass % of cationic surfactant, then, in the consideration, x+y mass % is to be used as a basis; by contrast, if e.g. x mass % of betaine surfactant and y mass % of nonionic surfactant are present, then merely the x mass % of the betaine surfactant is to be used as a basis.

Surfactants according to the invention may be: anionic surfactants, cationic surfactants and betaine surfactants. In each case, these may be branched or unbranched.

Surfactants generally consist of a hydrophobic moiety and of a hydrophilic moiety. Here, the hydrophobic moiety generally has a chain length of from 4 to 20 carbon atoms, preferably 6 to 19 carbon atoms and particularly preferably 8 to 18 carbon atoms. The functional unit of the hydrophobic group is generally an OH group, where the alcohol may be branched or unbranched. The hydrophilic moiety generally essentially consists of alkoxylated units (e.g. ethylene oxide (EO), propylene oxide (PO) and/or butylene oxide (BO), where usually 2 to 30, preferably 5 to 20, of these alkoxylated units are strung together, and/or charged units, such as sulfate, sulfonate, phosphate, carboxylic acids, ammonium and ammonium oxide.

Examples of anionic surfactants are: carboxylates, sulfonates, sulfo fatty acid methyl esters, sulfates, phosphates. Examples of cationic surfactants are: quaternary ammonium compounds. Examples of betaine surfactants are: alkylbetaines.

Here, a “carboxylate” is understood as meaning a compound which has at least one carboxylate group in the molecule. Examples of carboxylates which can be used according to the invention are
soaps—e.g. stearates, oleates, cocoates of the alkali metals or of ammonium,
ether carboxylates—e.g. Akypo® RO 20, Akypo® RO 50, Akypo® RO 90.

A “sulfonate” is understood as meaning a compound which has at least one sulfonate group in the molecule. Examples of sulfonates which can be used according to the invention are
alkylbenzenesulfonates—e.g. Lutensit® A-LBS, Lutensit® A-LBN, Lutensit® A-LBA, Marlon@ AS3, Maranil® DBS,
alkylsulfonates—e.g. Alscoap OS-14P, BIO-TERGE@ AS-40, BIO-TERGE® AS-40 CG, BIO-TERGE® AS-90 Beads, Calimulse® AOS-20, Calimulse® AOS-40, Calsoft® AOS-40, Colonial® AOS-40, Elfan® OS 46, Ifrapon® AOS 38, Ifrapon® AOS 38 P, Jeenate® AOS-40, Nikkol® OS-14, Norfox® ALPHA XL, POLYSTEP® A-18, Rhodacal® A-246L, Rhodacal® LSS-40/A,
sulfonated oils, such as, for example, Turkish red oil,
olefinsulfonates,
aromatic sulfonates—e.g. Nekal® BX, Dowfax® 2A1.

Here, a “sulfo fatty acid methyl ester” is understood as meaning a compound which has the following unit of the general formula (I):

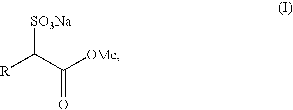

in which R has 10 to 20 carbon atoms; preferably, R has 12 to 18 and particularly preferably 14 to 16 carbon atoms.

Here, a “sulfate” is understood as meaning a compound which has at least one SO₄ group in the molecule. Examples of sulfates which can be used according to the invention are
fatty alcohol sulfates, such as, for example, coconut fatty alcohol sulfate (CAS 97375-27-4)—e.g. EMAL® 10G, Dispersogen® SI, Elfan® 280, Mackol® 100N,
other alcohol sulfates—e.g. Emal® 71, Lanette® E,
coconut fatty alcohol ether sulfate—e.g. Emal® 20C, Latemul® E150, Sulfochem® ES-7, Texapon® ASV-70 Spec., Agnique SLES-229-F, Octosol 828, POLYSTEP® B-23, Unipol® 125-E, 130-E, Unipol® ES-40,
other alcohol ether sulfates—e.g. Avanel® S-150, Avanel® S 150 CG, Avanel® S 150 CG N, Witcolate® D51-51, Witcolate® D51-53.

A “phosphate” is presently understood as meaning a compound which has at least one PO₄ group in the molecule. Examples of phosphates which can be used according to the invention are
alkyl ether phosphates—e.g. Maphos® 37P, Maphos® 54P, Maphos® 37T, Maphos® 210T and Maphos® 210P,
phosphates such as Lutensit A-EP,
alkyl phosphates.

A “quaternary ammonium compound” is understood as meaning a compound which has at least one R₄N⁺ group in the molecule. Examples of quaternary ammonium compounds which can be used according to the invention are
halides, methosulfates, sulfates and carbonates of coconut, tallow fatty or cetyl/oleyltrimethylammonium.

Furthermore, a “betaine surfactant” is understood as meaning a compound which, under application conditions, i.e. under standard pressure and at room temperature (20° C.) or under the conditions as have been chosen in the examples for the simulation, carries at least one positive and one negative charge. An “alkylbetaine” here is a betaine surfactant which has at least one alkyl unit in the molecule. Examples of betaine surfactants which can be used according to the invention are cocamidopropylbetaine—e.g. MAFO® CAB, Amonyl® 380 BA, AMPHOSOL® CA, AMPHOSOL® CG, AMPHOSOL® CR, AMPHOSOL® HCG; AMPHOSOL® HCG-50, Chembetaine® C, Chembetaine® CGF, Chembetaine® CL, Dehyton® PK, Dehyton® PK 45, Emery® 6744, Empigen® BS/F, Empigen® BS/FA, Empigen® BS/P, Genagen® CAB, Lonzaine® C, Lonzaine® CO, Mirataine® BET-C-30, Mirataine® CB, Monateric® CAB, Naxaine® C, Naxaine® CO, Norfox® CAPB, Norfox® Coco Betaine, Ralufon® 414, TEGO®-Betaine CKD, TEGO® Betaine E KE 1, TEGO®-Betaine F, TEGO®-Betaine F 50 and amine oxides, such as, for example, alkyldimethylamine oxides, i.e. compounds of the general formula (II)

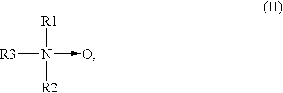

in which R1, R2 and R3, independently of one another, are an aliphatic, cyclic or tertiary alkyl or amidoalkyl radical, such as, for example Mazox® LDA, Genaminox®, Aromox® 14 DW 970.

A particularly preferred embodiment is the described composition in which the one or more N-alkylthiophosphoric triamide(s) is/are selected from the group consisting of: N-methylthiophosphoric triamide, N-ethylthiophosphoric triamide, N-propylthiophosphoric triamide (linear or branched), N-butylthiophosphoric triamide (linear or branched), N-pentylthiophosphoric triamide (linear or branched), N-hexylthiophosphoric triamide (linear or branched), N-cyclohexylthiophosphoric triamide, N-heptylthiophosphoric triamide (linear or branched), N-cycloheptylthiophosphoric triamide, N-octylthiophosphoric triamide (linear or branched), N-cyclooctylthiophosphoric triamide. Particular preference is given in this connection to those compositions which comprise at least one of the N-alkylthiophosphoric triamides selected from the group consisting of N-ethylthiophosphoric triamide, N-propylthiophosphoric triamide, N-butylthiophosphoric triamide and N-pentylthiophosphoric triamide. And very particular preference is given to compositions which comprise at least one of the N-alkylthiophosphoric triamides selected from the group consisting of N-propylthiophosphoric triamide and N-butylthiophosphoric triamide.

A further preferred embodiment of the present invention is a composition as described which comprises at least two N-alkylthiophosphoric alkylamides. Here, the most preferred embodiment is one in which the composition comprises N-propylthiophosphoric triamide and N-butylthiophosphoric triamide.

Since the N-alkylthiophosphoric alkylamides have reduced storage stability in the presence of strong acids and bases, preference is given to a composition which has a pH in the range from 5 to 9 and preferably from 6 to 8, such as 6.5, 7 or 7.5. However, the composition can also be used with strong acids or bases. In this connection, preference is given to using the dosing devices described in more detail below.

A further preferred embodiment of the present invention is a composition which additionally comprises at least one of the following substances: nonionic surfactant, polymer, dye, fragrance, complexing agent, acid, base, biocide, hydrotrope, thickener.

Nonionic surfactants are interface-active substances with a head group which is uncharged, does not carry an ion charge in the neutral pH range, is polar, hydrophilic and water-solubilizing (in contrast to anionic and cationic surfactants), and which adsorbs to interfaces and aggregates above the critical micelle concentration (cmc) to give neutral micelles. Depending on the type of hydrophilic head group, a distinction can be made between (oligo)oxyalkylene groups, in particular (oligo)oxyethylene groups (polyethylene glycol groups), which include the fatty alcohol polyglycol ethers (fatty alcohol alkoxylates), alkylphenol polyglycol ethers, and fatty acid ethoxylates, alkoxylated triglycerides and mixed ethers (polyethylene glycol ethers alkylated on both sides); and carbohydrate groups, which include, for example, the alkyl polyglucosides and fatty acid N-methylglucamides.

Examples of nonionic compounds are alcohol alkoxylates.

Alcohol alkoxylates are based on a hydrophobic moiety with a chain length of from 4 to 20 carbon atoms, preferably 6 to 19 carbon atoms and particularly preferably 8 to 18 carbon atoms, where the alcohol may be branched or unbranched, and a hydrophilic moiety, which may be alkoxylated units, e.g. ethylene oxide (EO), propylene oxide (PO) and/or butylene oxide (BuO), with 2 to 30 repeat units. Examples are, inter alia, Lutensol® XP, Lutensol® XL, Lutensol® ON, Lutensol® AT, Lutensol® A, Lutensol® AO, Lutensol® TO.

Alcohol phenol alkoxylates are compounds of the general formula (III),

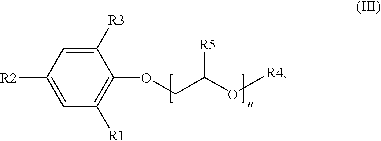

which are prepared by the addition of alkylene oxide, preferably of ethylene oxide, onto alkylphenols. Preferably, R4=H here. It is furthermore preferred if R5=H and is thus EO; if R5=CH3, it is PO and if R5=CH2CH3, it is BuO. Moreover, particular preference is given to a compound in which octyl-[(R1=R3=H, R2=1,1,3,3-tetramethylbutyl(diisobutylene)], nonyl-[(R1=R3=H, R2=1,3,5-trimethylhexyl(tripropylene)], dodecyl-, dinonyl- or tributylphenol polyglycol ethers (e.g. EO, PO, BuO) R—C6H4-O-(EO/PO/BuO)n R C8 to C12, where n=5 to 10, are present. Nonexhaustive examples of such compounds are: Norfox® OP-102, Surfonic® OP-120, T-Det® O-12.

Fatty acid ethoxylates are fatty acid esters after-treated with varying amounts of ethylene oxide (EO).

Triglycerides are esters of glycerol (glycerides) in which all three hydroxy groups are esterified with fatty acids. These can be modified with alkylene oxide.

Fatty acid alkanolamides are compounds of the general formula (IV)

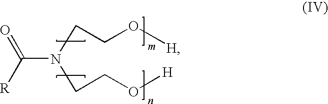

which has at least one amide group with an alkyl radical R and one or two alkoxy radical(s), where R comprises 11 to 17 carbon atoms and 1≦m+n≦5.

Alkyl polyglycosides are mixtures of alkyl monoglucoside(alkyl-α-D- and -β-D-glucopyranoside, and small fractions of -glucofuranoside), alkyl diglucosides (-isomaltosides, -maltosides and others) and alkyl oligoglucosides (-maltotriosides, -tetraosides and others). Alkyl polyglycosides are accessible, inter alia, by acid-catalyzed reaction (Fischer reaction) from glucose (or starch) or from n-butyl glucosides with fatty alcohols. Alkyl polyglycosides correspond to the general formula (V)

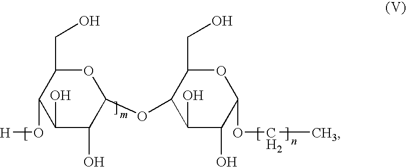

in which

m=0 to 3 and

n=4 to 20.

One example is Lutensol® GD70.

In the group of nonionic /alkylated, preferably N-methylated, fatty acid amides of the general formula (VI)

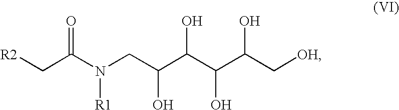

R1 is usually an n-C₁₂-alkyl radical, R2 is an alkyl radical having 1 to 8 carbon atoms. R2 is preferably methyl.

The advantage of adding these nonionic surfactants is that they lower the interfacial tension and thus ensure good wetting.

Polymers may be: adducts consisting of ethylene oxide (EO) and/or propylene oxide (PO) and/or butylene oxide (BuO). The arrangement of the monomers here may be alternating, random or blockwise. Preference is given to compounds in which the distribution is essentially blockwise. Examples of such compounds are Pluronics®.

Dyes may be, inter alia: Acid Blue 9, Acid Yellow 3, Acid Yellow 23, Acid Yellow 73, Pigment Yellow 101, Acid Green 1, Acid Green 25. The advantage of using dyes in cleaners is that they facilitate dosing and, if appropriate, give an indication during cleaning of where cleaning has already taken place.

Fragrances may be individual compounds or mixtures of alcohols, aldehydes, terpenes and/or esters. Examples of fragrances are: lemongrass oil, cochin, dihydromyrcenol, lilial, phenylethyl alcohol, tetrahydrolinalool, hexenol cis-3, lavandin grosso, citral, allyl caproate, citronitrile, benzyl acetate, hexylcinnamaldehyde, citronellol, isoamyl salicylate, isobornyl acetate, terpinyl acetate, linalyl acetate, terpinyl acetate, agrunitrile, eucalyptus oil, herbaflorat and orange oil. The advantage of using fragrances in cleaners is that, during cleaning, they give an indication of where cleaning has already taken place, and also increase the perception of the cleaning effect of the composition to a level other than a visual level.

Complexing agents are compounds which are able to bind cations. This can be utilized in order to reduce the hardness of water and in order to precipitate out troublesome heavy metal ions. Examples of complexing agents are NTA, EDTA, MGDA and GLDA. The advantage of using these compounds is that many cleaning-active compounds achieve a better effect in soft water; furthermore, by reducing the water hardness, it is possible to avoid the occurrence of lime deposits after cleaning. The use of these compounds thus dispenses with the need to dry the cleaned surface. From the point of view of process flow, this is advantageous and in particular consequently desirable since, in this way, the composition according to the invention applied for the preservation is not partially removed again.

Acids are compounds which are advantageously used to dissolve lime deposits. Examples of acids are formic acid, acetic acid, citric acid, hydrochloric acid, sulfuric acid and sulfonic acid.

Bases are compounds which can advantageously be used for establishing the favorable pH range for complexing agents. Examples of bases which can be used according to the invention are: NaOH, KOH and aminoethanol.

Biocides are compounds which kill bacteria. One example of a biocide is glutaraldehyde. The advantage of using biocides is that they counteract the spread of pathogens.

Hydrotropes are compounds which improve the solubility of the surfactant/surfactants in the composition. One example of a hydrotrope is: cumene sulfonate.

Thickeners are compounds which increase the viscosity of the composition. Examples of thickeners are: e.g. polyacrylates or hydrophobically modified polyacrylates. The advantage of using thickeners is that liquids with a higher viscosity have a higher residence time on inclined or vertical surfaces than do liquids with a lower viscosity. This increases the interaction time between composition and surface to be cleaned.

A dosing device for the composition according to the invention is further provided by the present invention. A dosing device within the context of this invention is a vessel which comprises the composition according to the invention and releases it through at least one opening. Here, the removal can take place as a result of the force of gravity, e.g. by pouring out through an opening, by pumping, e.g. by generating a superatmospheric pressure in the vessel, or else by applying a subatmospheric pressure from outside. It is also advantageous to convey the composition according to the invention from the container with the help of a propellant gas. In this connection, preference is given to those dosing devices which distribute the composition according to the invention as homogeneously as possible on the surface to be treated or on the cleaning device to be used for the cleaning. Particular preference is given here to a dosing device in which at least two of the constituents of the composition according to the invention are only mixed with one another at the point of delivery. This type of dosing device is particularly advantageous when, besides the at least one N-alkylthiophosphoric triamide, one or more surfactants are used which are especially acidic or basic. If further constituents are acids or bases, it is particularly advantageous to separate these and also to separate these from the N-alkylthiophosphoric triamide(s) during storage and only to combine the constituents upon use.

A kit of parts consisting of at least two substances that are to be used simultaneously or successively which together correspond to the composition according to the invention is further provided by the present invention. Thus, for example, the one or more N-alkylthiophosphoric triamide(s) may be present in one container, and the one or more surfactants may be present in a second container. Separation into strongly acidic and/or strongly basic constituents on the one hand and the one or more N-alkylthiophosphoric triamide(s) on the other hand can thus also be realized and lies within the scope of the present invention. Besides allowing the essentially simultaneous use of the various constituents, such a kit of parts also allows a staggered use of the constituents. Thus, for example, firstly the cleaning of the surface to be cleaned can take place with the surfactant-containing composition, followed by the preservation of the surface with the composition comprising N-alkylthiophosphoric triamide(s). Finally, it is also possible to use two compositions where the first comprises surfactant(s) and N-alkylthiophosphoric triamide(s) and the second comprises only N-alkylthiophosphoric triamide(s). In this way, the odor nuisance during cleaning with the first composition can be reduced, and a preservation can then be achieved with the second composition. This too forms a preferred subject matter of the present invention.

A cleaning device which has the composition according to the invention is further provided by the present invention. A cleaning device within the context of this invention is any which is suitable for achieving a cleaning effect. This includes, inter alia: sponges, cloths, wipes, wipers made of metal, plastic, glass, ceramic and/or rubber, nonwovens and brushes.

The use of a composition according to the invention, of a dosing device according to the invention, of a kit of parts according to the invention and/or of a cleaning device according to the invention for the cleaning, preferably for the cleaning of hard surfaces and/or materials and/or upholstery is further provided by the invention.

A use as described in which the surface to be cleaned is selected from the group consisting of tiles, marble, ceramic, concrete, plastic, metal, enamel, glass is a preferred subject matter of the invention. A use in which the soiling to be removed contains urine and/or its degradation products is likewise a preferred embodiment.

The present invention is illustrated in more detail below by examples:

### EXAMPLES

1.0 mass % or 5.0 mass % of NxPT (3:1 N-(n-butyl)thiophosphonic triamide:N-propylthiophosphoric triamide) were added to a cleaner in each case. An ammonia test was then carried out with the samples, i.e. both with the cleaner per se, and also with the two NxPT-containing compositions, the “cleaner mixture”.

Ammonia Test:
250 mg of this cleaner mixture were weighed into a 100 ml Erlenmeyer flask.
30 mg of solid urease (from sword beans; lyophilized 5 U/mg for determining the urea in the serum, Merck, Article No. 4194753) were weighed into a 100 ml beaker
50 ml of 0.9% strength sodium chloride solution which comprised urea (8.56 g/l) were added to the urease
the entire liquid from the beaker was poured quickly into the sample in the Erlenmeyer flask
a diffusion tube (Drager tube, ammonia 20/a-D, 20-1500 ppm*h, order No. 8101301) was attached such that the flask was closed (tube in a bored rubber stopper)
every 30 minutes the value of the diffusion tube was read off and noted; measurement lasted for a total of 6 hours

Double Determination (Value 1, Value 2)

### Example 1

No cleaner

No cleaner

| Sample No. | NH₃ in ppm * h | | | |
|---|---|---|---|---|
| Blank value | Time (h) | Value 1 | Value 2 | Average value (AV) |
| | 0.5 | 225 | | 225 |
| | 1 | 550 | | 550 |
| | 1.5 | 1000 | | 1000 |
| | 2 | <1500 | | 1500 |
| | 2.5 | | | |
| | 3 | | | |
| | 3.5 | | | |
| | 4 | | | |
| | 4.5 | | | |
| | 5 | | | |
| | 5.5 | | | |
| | 6 | | | |

### Examples 2-4

Form 1 was a cleaner formulation consisting of:

| | | |
|---|---|---|
| Dodecylbenzene sulfonate, amine salt | 3.3 | mass % |
| C13,15-oxo alcohol + 8 EO | 3 | mass % |
| Alkyl polyglucoside | 1 | mass % |
| Water | 94.7 | mass % |

| Sample No. | | NH3 in ppm * h | | | |
|---|---|---|---|---|---|
| Form 1 | No PxPT | Time (h) | Value 1 | Value 2 | AV |
| | | 0.5 | 190 | 150 | 170 |
| | | 1 | 500 | 400 | 450 |
| | | 1.5 | 900 | 750 | 825 |
| | | 2 | 1400 | 1150 | 1275 |
| | | 2.5 | <1500 | <1500 | 1500 |
| | | 3 | | | |
| | | 3.5 | | | |
| | | 4 | | | |
| | | 4.5 | | | |
| | | 5 | | | |
| | | 5.5 | | | |
| | | 6 | | | |

| Sample No. | | NH3 in ppm * h | | | |
|---|---|---|---|---|---|
| Form 1 | 1.0% NXPT | Time (h) | Value 1 | Value 2 | AV |
| | | 0.5 | 140 | 165 | 152.5 |
| | | 1 | 375 | 395 | 385 |
| | | 1.5 | 625 | 650 | 637.5 |
| | | 2 | 950 | 975 | 962.5 |
| | | 2.5 | 1300 | 1300 | 1300 |
| | | 3 | <1500 | <1500 | 1500 |
| | | 3.5 | | | |
| | | 4 | | | |
| | | 4.5 | | | |
| | | 5 | | | |
| | | 5.5 | | | |
| | | 6 | | | |
| Sample No. | | NH3 in ppm * h | | | |
| Form 2 | 5.0% NXPT | Time (h) | Value 1 | Value 2 | AV |
| | | 0.5 | 80 | 75 | 77.5 |
| | | 1 | 180 | 175 | 177.5 |
| | | 1.5 | 250 | 250 | 250 |
| | | 2 | 350 | 350 | 350 |
| | | 2.5 | 480 | 480 | 480 |
| | | 3 | 600 | 590 | 595 |
| | | 3.5 | 700 | 690 | 695 |
| | | 4 | 850 | 800 | 825 |
| | | 4.5 | 1000 | 980 | 990 |
| | | 5 | 1150 | 1100 | 1125 |
| | | 5.5 | 1300 | 1300 | 1300 |
| | | 6 | 1500 | 1450 | 1475 |

### Examples 5-7

Form 2 was a cleaner formulation consisting of:

| | | |
|---|---|---|
| C₁₂-C₁₈-Alcohol + 1-8 PO + 1-8 EO + | 6 | mass % |
| C₈-C₁₈-alkyl polyglucoside + C₈-C₁₀-carboxylic | | |
| acid | | |
| Penta-sodium triphosphate | 3 | mass % |
| Butyl glycol | 10 | mass % |
| Water | 81 | mass %. |

| | |
|---|---|
| Sample No. | NH3 in ppm * h |

| Form 2 | No NxPT | Time (h) | Value 1 | Value 2 | AV |
|---|---|---|---|---|---|
| | | 0.5 | 130 | 130 | 130 |
| | | 1 | 400 | 425 | 412.5 |
| | | 1.5 | 790 | 800 | 795 |
| | | 2 | 1250 | 1300 | 1275 |
| | | 2.5 | <1500 | <1500 | 1500 |
| | | 3 | | | |
| | | 3.5 | | | |
| | | 4 | | | |
| | | 4.5 | | | |
| | | 5 | | | |
| | | 5.5 | | | |
| | | 6 | | | |
| Sample No. | | NH3 in ppm * h | | | |
| Form 2 | 1.0% NXPT | Time (h) | Value 1 | Value 2 | AV |
| | | 0.5 | 10 | 20 | 15 |
| | | 1 | 40 | 35 | 37.5 |
| | | 1.5 | 60 | 50 | 55 |
| | | 2 | 80 | 75 | 77.5 |
| | | 2.5 | 100 | 95 | 97.5 |
| | | 3 | 130 | 120 | 125 |
| | | 3.5 | 175 | 150 | 162.5 |
| | | 4 | 200 | 190 | 195 |
| | | 4.5 | 220 | 200 | 210 |
| | | 5 | 270 | 250 | 260 |
| | | 5.5 | 300 | 290 | 295 |
| | | 6 | 350 | 300 | 325 |
| Sample No. | | NH3 in ppm * h | | | |
| Form 2 | 5.0% NXPT | Time (h) | Value 1 | Value 2 | AV |
| | | 0.5 | 20 | 20 | 20 |
| | | 1 | 40 | 40 | 40 |
| | | 1.5 | 50 | 55 | 52.5 |
| | | 2 | 75 | 80 | 77.5 |
| | | 2.5 | 100 | 100 | 100 |
| | | 3 | 120 | 120 | 120 |
| | | 3.5 | 140 | 150 | 145 |
| | | 4 | 190 | 180 | 185 |
| | | 4.5 | 200 | 200 | 200 |
| | | 5 | 240 | 220 | 230 |
| | | 5.5 | 290 | 250 | 270 |
| | | 6 | 300 | 300 | 300 |

### Examples 8-10

Form 3 was a dilution of form 1 in the ratio 1:20.

| Sample No. | | NH3 in ppm * h | | | |
|---|---|---|---|---|---|
| Form 3 | No NxPT | Time (h) | Value 1 | Value 2 | AV |
| | | 0.5 | 320 | 300 | 310 |
| | | 1 | 900 | 900 | 900 |
| | | 1.5 | 1500 | 1500 | 1500 |
| | | 2 | | | |
| | | 2.5 | | | |
| | | 3 | | | |
| | | 3.5 | | | |
| | | 4 | | | |
| | | 4.5 | | | |
| | | 5 | | | |
| | | 5.5 | | | |
| | | 6 | | | |

| Sample No. | | NH3 in ppm * h | | | |
|---|---|---|---|---|---|
| Form 3 | 1.0% NXPT | Time (h) | Value 1 | Value 2 | AV |
| | | 0.5 | 100 | 125 | 112.5 |
| | | 1 | 300 | 325 | 312.5 |
| | | 1.5 | 550 | 560 | 555 |
| | | 2 | 800 | 820 | 810 |
| | | 2.5 | 1100 | 1200 | 1150 |
| | | 3 | 1500 | <1500 | 1500 |
| | | 3.5 | <1500 | | 1500 |
| | | 4 | | | |
| | | 4.5 | | | |
| | | 5 | | | |
| | | 5.5 | | | |
| | | 6 | | | |
| Sample No. | | NH3 in ppm * h | | | |
| Form 3 | 5.0% NXPT | Time (h) | Value 1 | Value 2 | AV |
| | | 0.5 | 75 | 75 | 75 |
| | | 1 | 180 | 175 | 177.5 |
| | | 1.5 | 270 | 280 | 275 |
| | | 2 | 375 | 375 | 375 |
| | | 2.5 | 500 | 500 | 500 |
| | | 3 | 625 | 625 | 625 |
| | | 3.5 | 780 | 790 | 785 |
| | | 4 | 900 | 900 | 900 |
| | | 4.5 | 1050 | 1000 | 1025 |
| | | 5 | 1200 | 1150 | 1175 |
| | | 5.5 | 1400 | 1300 | 1350 |
| | | 6 | <1500 | 1500 | 1500 |

### Examples 11-13

Form 4 was a cleaner consisting of:

| | | |
|---|---|---|
| C13,15-Oxo alcohol + 7 EO | 5 | mass % |
| Coconut alkali metal soap | 1.8 | mass % |
| Dodecylbenzenesulfonate, amine salt | 8.25 | mass % |
| Penta-sodium triphosphate | 3 | mass % |
| Cumene sulfonate | 3.2 | mass % |
| Water | 78.75 | mass % |

| Sample No. | | NH3 in ppm * h | | | |
|---|---|---|---|---|---|
| Form 4 | No NxPT | Time (h) | Value 1 | Value 2 | AV |
| | | 0.5 | 290 | 310 | 300 |
| | | 1 | 850 | 950 | 900 |
| | | 1.5 | 1500 | 1500 | 1500 |
| | | 2 | | | |
| | | 2.5 | | | |
| | | 3 | | | |
| | | 3.5 | | | |
| | | 4 | | | |
| | | 4.5 | | | |
| | | 5 | | | |
| | | 5.5 | | | |
| | | 6 | | | |

| Sample No. | | NH3 in ppm * h | | | |
|---|---|---|---|---|---|
| Form 4 | 1.0% NXPT | Time (h) | Value 1 | Value 2 | AV |
| | | 0.5 | 90 | 100 | 95 |
| | | 1 | 250 | 250 | 250 |
| | | 1.5 | 450 | 470 | 460 |
| | | 2 | 650 | 660 | 655 |
| | | 2.5 | 890 | 875 | 882.5 |
| | | 3 | 1150 | 1125 | 1137.5 |
| | | 3.5 | 1375 | 1350 | 1362.5 |
| | | 4 | <1500 | <1500 | 1500 |
| | | 4.5 | | | |
| | | 5 | | | |
| | | 5.5 | | | |
| | | 6 | | | |

| Sample No. | | NH3 in ppm * h | | | |
|---|---|---|---|---|---|
| Form 4 | 5.0% NXPT | Time (h) | Value 1 | Value 2 | AV |
| | | 0.5 | 50 | 50 | 50 |
| | | 1 | 100 | 100 | 100 |
| | | 1.5 | 150 | 160 | 155 |
| | | 2 | 225 | 225 | 225 |
| | | 2.5 | 300 | 300 | 300 |
| | | 3 | 390 | 385 | 387.5 |
| | | 3.5 | 480 | 480 | 480 |
| | | 4 | 550 | 500 | 525 |
| | | 4.5 | 650 | 600 | 625 |
| | | 5 | 790 | 700 | 745 |
| | | 5.5 | 850 | 800 | 825 |
| | | 6 | 980 | 850 | 915 |

The results show that when using the compositions according to the invention, the release of ammonia, which is responsible for the urine odor, was significantly reduced compared to all cleaners.

1. A composition comprising at least one N-alkylthiophosphoric triamide and at least one surfactant selected from the group consisting of anionic surfactants, cationic surfactants and betaine surfactants.

2. The composition according to claim 1, in which the total amount of N-alkylthiophosphoric triamide(s) is 0.01 to 75 mass %.

3. The composition according to claim 1, in which the total amount of N-alkylthiophosphoric triamide(s) is 0.1 to 50 mass %.

4. The composition according to claim 3, in which the total amount of N-alkylthiophosphoric triamide(s) is 0.25 to 20 mass %.

5. The composition according to claim 3, in which the total amount of N-alkylthiophosphoric triamide(s) is 0.5 to 10 mass %.

6. The composition according to claim 1, in which the at least one surfactant is present in a total amount of from 0.01 to 99 mass %.

7. The composition according to claim 1, in which the at least one surfactant is present in a total amount of from 0.5 to 50 mass %.

8. The composition according to claim 1, in which the at least one surfactant is present in a total amount of from 1 to 25 mass %.

9. The composition according to claim 1, in which the at least one surfactant is present in a total amount of from 1 to 15 mass %.

10. The composition according to claim 1, in which the one or more N-alkylthiophosphoric triamide(s) is/are selected from the group consisting of: methylthiophosphoric triamide, ethylthiophosphoric triamide, N-propylthio-phosphoric triamide (linear or branched), N-butylthiophosphoric triamide (linear or branched), N-pentylthiophosphoric triamide (linear or branched), N-hexylthio-phosphoric triamide (linear or branched), N-cyclohexylthiophosphoric triamide, N-heptylthiophosphoric triamide (linear or branched), N-cycloheptylthiophosphoric triamide, N-octylthiophosphoric triamide (linear or branched), and N-cyclooctylthio-phosphoric triamide.

11. The composition according to claim 1, which comprises at least two N-alkylthiophosphoric triamides.

12. The composition according to claim 1, which has a pH in the range from 5 to 9.

13. The composition according to claim 12, which has a pH of from 6 to 8.

14. The composition according to any one of claims 1 to 13, which comprises at least one of the substances selected from the group consisting of: nonionic surfactant, dye, fragrance, acid, base, complexing agent, biocide, hydrotrope, and thickener.

15. A dosing device for a composition according to claim 1.

16. The dosing device in which at least two of the constituents of the composition according to claim 1 are only mixed with one another at the point of delivery.

17. A kit of parts consisting of at least two substances to be used simultaneously or successively which together correspond to the composition according to claim 1.

18. A cleaning device which has the composition according to claim 1.

19. A cleaner of hard surfaces and/or materials and/or upholstery comprising a composition according to claim 1.

20. The cleaner according to claim 19, in which the surface to be cleaned is selected from the group consisting of tiles, marble, ceramic, concrete, plastic, metal, enamel, and glass.
